(19) United States
(12) Patent Application Publication
(10) Pub. No.: US 2023/0226172 A1
(43) Pub. Date: Jul. 20, 2023

(54) Inactivation of Genome Enveloped within Coronavirus Spherical or Pleomorphic Particles or Shells to Form a Vaccine

(71) Applicants: Schossau M. Tom, Bronxville, NY (US); Kurup K. Shree, Solon, OH (US); Schossau Jorden, Mason, MI (US)

(72) Inventors: Schossau M. Tom, Bronxville, NY (US); Kurup K. Shree, Solon, OH (US); Schossau Jorden, Mason, MI (US)

(21) Appl. No.: 18/009,851

(22) PCT Filed: Jun. 11, 2021
(86) PCT No.: PCT/US2021/037090

§ 371 Date: Dec. 12, 2022

Related U.S. Application Data
(60) Provisional application No. 63/038,161, filed on Jun. 12, 2020.

(51) Int. Cl. A61K 39/215

(52) U.S. Cl. CPC A61K 39/215; A61K 2039/5252

(57) ABSTRACT

Vaccine based on ethanol inactivated pathogens, or part thereof, are described herein. Also disclosed are certain vaccines for treating COVID-19 or other coronavirus related diseases is created by deactivating the genome, genetic material or RNA encapsulated within the shell of virus without eliminating the spikes or spike protein, which both attaches the virus to a host cell and is detected by the body to produce antibodies. Treatment of an active coronavirus with a material such as an effective amount of ethanol will both penetrate the shell and deactivate the genetic material which causes the disease, for preparation of a vaccine.

### CROSS REFERENCE TO RELATED APPLICATIONS

This Application claims priority to U.S. Provisional Application Serial No. 63038161, filed on Jun. 12, 2020, the entire contents of which is incorporated herein by reference thereto.

### FIELD OF THIS INVENTION

This invention is directed to the inactivation of the coronavirus that causes COVID-19, SARS-CoV-2 and to other coronaviruses or similar viruses. The invention further provides compositions and methods for generation and administering novel vaccines.

### BACKGROUND OF THE INVENTION

### Vaccination and Immune Response

When an individual first encounters a new pathogen (such as a bacteria, fungus, virus, protozoa, or algae) it can take several days to mount a response to the challenge. Typically, the immune system will make and use all the germ-fighting tools it requires to contain and/or eliminate the infection. After the infection, the immune system maintains a few T-lymphocytes, called memory cells, that go into action quickly if the body encounters the same pathogen again. When antigens associated with the pathogen are detected again, B-lymphocytes are recruited and expanded to produce antibodies to attack them.

Vaccines are used to jumpstart immunity by presenting pathogenic antigens thereby imitating an infection. The goal of this mimicry is to cause the immune system to produce T-lymphocytes and antibodies against the pathogen without causing the related illness. Sometimes, after getting a vaccine, the imitation infection can cause minor symptoms, such as fever. Such minor symptoms are typically generated by the activation of the body’s immune system and are not unusual as the body builds immunity.

Once challenged, the body maintains a reservoir of “memory” T-lymphocytes, as well as B-lymphocytes with specificity for that disease in the future, but it typically takes a few weeks for the body to produce T-lymphocytes and B-lymphocytes after vaccination.

### Vaccine Types

There are various approaches to developing vaccines. These approaches are based on information about pathogen type (e.g., virus or bacteria), route of infection, and the parameters of subsequent immune respond. In addition, other considerations such as geographic location of outbreak, medical infrastructure, and economics, are also important. There are various types of vaccines in use/development today.

Live, attenuated vaccines are used for viruses and bacteria. These vaccines contain a version of the living virus or bacteria that has been weakened so that it does not cause serious disease in people with healthy immune systems. However, although live, attenuated vaccines are considered very effective, not everyone can receive this type of vaccine. For example, individuals with diminished immune systems should not be given live vaccines.

Inactivated pathogen vaccines are also used against viruses and bacteria. Or this type of vaccine the pathogen is inactivated, or killed, prior to being incorporated into the vaccine and then presented to challenge the body’s immune system. The inactivated polio vaccine is an example of this type of vaccine. Inactivated vaccines produce immune responses in different ways than live, attenuated vaccines. Often, multiple doses are necessary to build up and/or maintain immunity.

Subunit vaccines only use parts/subunits of the pathogen to challenge the recipients immune system instead of the entire pathogen. Presenting only a part or subunit of a pathogen in these types of vaccines can be accomplished in several ways. For example, specific pathogen subunits may be isolated from the remainder of the organism then incorporated into the vaccine. Alternatively, recombinant DNA or RNA vaccines may be used to generate and present a subunit/part of a pathogen to the recipient’s immune system. Instead of injecting a weakened form of a virus or bacteria into the body as with a traditional vaccine, DNA and RNA vaccines use part of the virus’ own genetic code to stimulate an immune response.

Toxoid vaccines prevent diseases caused by bacteria that produce toxins (poisons) in the body. In these vaccines, the toxins are weakened so they cannot cause illness-these weakened toxins are called toxoids.

### Vaccine Compositions

Modern vaccines typically incorporate combinations of some or all of the following components: pathogen antigens or a means of generating pathogen antigens, stabilizers, adjuvants, antibiotics, and preservatives.

Preservatives are used to prevent contamination. Adjuvants such as Aluminum salts are used to help boost the body’s immune response to the vaccine. Adjuvants are known to exert their effect through different mechanisms. For example Aluminums and emulsions generate depots that trap antigens at the injection site for a sustained stimulation of the immune system through an increased recruitment and activation of antigen presenting cells (APCs). PRR agonists adjuvants usually include substances that stimulate pattern recognition receptors (PRR), which are essential components of innate immunity required for activating antigen-presenting cells (APC) and serve as a bridge between innate and adaptive immunity. Almost all PRRs are potential targets for adjuvants. Stabilizers are used to keep the vaccine effective after manufactured. Residual cell culture material may be present in some vaccines. These materials come from the cell cultures used to grow the pathogen, or parts thereof, used in the vaccine. Vaccines may also contain residual inactivating ingredients. For example chemicals such as Formaldehyde may be used to kill viruses or inactivate toxins during the vaccine manufacturing process. Vaccines may also contain residual antibiotics that were used prevent contamination by bacteria during the vaccine manufacturing process.

### Vaccine Administration Routes

Vaccines may be administered to individual recipients via various routes of administration. See e.g., https://www.cdc.gov/vaccines/hcp/acip-recs/general-recs/ administration.html

Some vaccines may be administered by subcutaneous, intradermal, or intramuscular injection. The method of administration of injectable vaccines is determined, in part, by the inclusion of adjuvants in some vaccines. An adjuvant is a vaccine component distinct from the antigen that enhances the immune response to the antigen, but might also increase risk of adverse reactions. To decrease risk of local adverse events, inactivated vaccines containing an adjuvant should be injected into a muscle. Administering a vaccine containing an adjuvant either subcutaneously or intradermally can cause local irritation, induration, skin discoloration, inflammation, and granuloma formation.

Still other vaccines are administered orally. For example, rotavirus, adenovirus, cholera vaccine, and oral typhoid vaccines have been administered orally. In certain instances, vavvines have been administered via an intranasal application. For example, live attenuated influenza vaccine has been approved in the United States. The administration device is may be a nasal sprayer with a dose-divider mechanism.

### COVID-19 Virus

As understood coronaviruses in general cause acute upper and lower_respiratory infections which can be mild infections, such as the common cold, or more serious and life threatening infections, such as COVID-19. The major difference between coronaviruses that cause a cold and those that cause a severe illness is that the former primarily infect the upper respiratory tract (the nose and throat), whereas the latter thrive in the lower respiratory tract (the lungs) and can lead to pneumonia. Coronaviruses are classified together on the basis of the crown or halo-like appearance of the envelope glycoproteins, and on characteristic features of chemistry and replication.

A coronavirus particle consists of four structural proteins: the nucleocapsid, envelope, membrane and spike. The nucleocapsid forms the genetic core, encapsulated in a ball formed by the envelope and membrane proteins within a lipid outer layer membrane.

The structure of a coronavirus generally comprises spherical or pleomorphic particles containing single stranded (positive-sense) RNA associated with nucleoprotein within a capsid comprised of a matrix protein. A lipid bilayer envelope contains interspersed envelope and membrane proteins (that makes a shell), is studded with projecting club-shaped glycoprotein projections (or spikes) and surrounds a core consisting of matrix protein enclosed within which are the single strand RNA (Mr 6 × 10⁶) associated with nucleoprotein. The glycoprotein spikes are responsible for attachment to the host cell and also carry the main antigenic epitopes, particularly the epitopes recognized by neutralizing antibodies.

To multiply coronaviruses enter the host cells, and the uncoated genome is transcribed and translated. New virions form by budding from host cell membranes. It is thought that human coronaviruses enter cells, predominately by (ACE-2) specific receptors.

Studies in both organ cultures and human volunteers show that earlier coronaviruses are extremely fastidious and grow only in differentiated respiratory epithelial cells. Infected cells become vacuolated, show damaged cilia and may form syncytia. Cell damage triggers production of inflammatory mediators, which cause local inflammation and swelling.

Host defenses have been studied for coronaviruses known before the discovery of the coronavirus that causes COVID-19, SARS-CoV-2. As understood for these earlier and sometimes milder versions, mucociliary activity is designed to clear the airways of particulate material, but coronaviruses can successfully infect the superficial cells of the ciliate epithelium. Only about one-third to one-half of individuals infected by these earlier coronaviruses develop symptoms. Because these earlier coronaviruses are common, many individuals have specific antibodies that can protect against infection. Most of these antibodies are directed against the surface projections and neutralize the infectivity of the virus.

In essence, scout-type T cells recognize the foreign body spikes, and produce cytokines that call_for special killer T cells to eradicate the foreign body. The biggest problem with these SARS-type viruses is thought to be that the immune system “overreacts,” calling for too many killer T-cells that then creates collateral damage-the cytokine storm creates excessive fluid that is incompatible for human life.

One earlier coronavirus that was more serious than the coronavirus associated with the common cold is the virus that causes Sever Acute Respiratory Syndrome (“SARS”). To infect a human host, this SARS coronavirus gains entry into individual human cells by attaching to cell surface protein then uses the cells’ internal machinery to produce copies of themselves, which subsequently spill out and spread to new cells. The molecular key to the SARS Cov-2 has been determined to be a spike protein, or S-protein. Research revealed that the SARS Cov-2 coronavirus attaches to a receptor on respiratory cells called angiotensin-converting enzyme 2 or ACE-2. It was found that the molecular bond between SARS-CoV-2′s spike protein and ACE-2 looks fairly similar to the binding structure of the coronavirus that caused the outbreak of SARS in 2003. There are differences in the precise amino acids used to bind SARS-CoV-2 to that ACE-2 receptor compared with the virus that causes SARS or acute respiratory syndrome. It is believed that there may differences between structure of the spikes associated with SARS and the spikes associated with COVID-19. It is understood that currently available vaccines modify the current spike codes for SARS-CoV and to artificially manufacture spike codes for SARS-CoV-2.

Viruses typically invade a cell, use its components to replicate and then infect other cells. RNA replication typically lacks the error-correction mechanism when copying DNA, and because coronaviruses have the longest RNA genomes and mutate very rapidly, there will be statistically more errors as mutations that may have new characteristics such as being more virulent or contagious.

Coronaviruses have two proteins that are prone to mutations - the spike protein and “accessory” proteins that are not fully understood, but essentially react to shut down the host’s immune response. Consequently, these mutations in coronaviruses aid in their evolution, and survival by evading the immune responses in various hosts.

There are concerns about the effectiveness of potential coronavirus vaccines, and the object of this invention is to provide an approach the problem in a manner that would not be subject to the same concerns or potential problems.
(1) The durability or long-term immunity may not develop, especially if COVID-19 acts like other coronaviruses (with mutations, where new vaccines must then be constantly made to counter the mutations).
(2) Current vaccine initiatives artificially make the spike proteins by “reverse engineering” the protein codes, but this concept has not been proven in humans.

Scientists still do not fully understand key aspects of COVID-19 that includes how the immune system will respond once exposed to the live coronavirus and especially, artificially made spike proteins introduced as a vaccine. Consequently, inactivating the coronavirus while keeping the spike proteins biologically intact will conceptually overcome these major concerns with current vaccine initiatives while reducing manufacturing time, difficulties and costs.

Recombinant replication of RNA/DNA proteins, or antigens, of spikes, the current focus by pharmaceuticals, should not cause infections, but have yet to be proven effective in humans, and manufacturing takes time if of the essence. Attenuating the virus, but which takes time to develop thru injecting/reinjecting into animals is one option by which a vaccine may be developed. Inactivated vaccines are attractive because they are readily prepared and capable of presenting an antigenic moiety similar to what the immune system would encounter in invading pathogens. Current vaccines that replicate the DNA or RNA codes needed for the immune response must be constantly remade/retested after viral mutations, however, a viral inactivation process (with ethanol) as described herein would be an effective vaccine against subsequent mutations (based on broader spectrum of pathogen epitope presentation to the recipient’s immune system) as well as be readily repeatable for new variants.

### BRIEF SUMMARY OF SOME OF THE PREFERRED EMBODIMENTS

Presented are methods of generating a vaccine against a pathogen that include: obtaining a sample of the pathogen; and treating the pathogen sample with ethanol to inactive or impair the pathogen. In certain embodiments, these methods include treating the pathogen with ethanol at a concentration of between about 10% to about 80% weight for a period of about 30 seconds. In serval of these embodiments, the pathogen presented is a corona virus and in some of these vaccines the pathogen targeted is the corona virus that causes Covid-19.

Still other embodiments, provide a vaccine composition having an ethanol inactivated or impaired pathogen; and a pharmaceutically acceptable carrier. Some of these embodiments may also include an adjuvant, stabilizer, antibiotic, gels, protective coatings, preservatives, residual cell cultures particles, and the like.

Other embodiments of the present invention provide methods for vaccinating an organism against a pathogen that include obtaining a vaccine comprising a whole pathogen that has been ethanol inactivated or impaired. In some cases the pathogen is rendered replication incompetent and/or there is a functionally disrupt any lipid bilayers.

The foregoing has outlined rather broadly the features and technical advantages of the present invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other embodiments for carrying out the same purposes of the present invention. It should also be realized by those skilled in the art that such equivalent embodiments do not depart from the spirit and scope of the invention as set forth in the appended claims.

### BRIEF DESCRIPTION OF FIGURES

FIG. 1 depicts a viral envelop, spike protein, and lipid bilayer disruption.

### DETAILED DESCRIPTION OF THE PREFERRED EMBODIMENTS

As used in this disclosure the term “pathogen” means any microorganism that can cause disease or other disorder, including but not limited to bacteria, fungi, viruses, protozoa, algae, and by-products thereof. The vaccines disclosed herein are intended for the treatment of any relevant living organism, including but not limited to humans, animals, and plants.

Studies have shown UV inactivation of SARS-CoV have created antibodies in mice. Inactivating the virus by destroying the viral shell or equivalently cutting off the spikes which will not create an infection, but is fully expected to create immunity in humans. Disrupting the inside “critical genomic mass” directly appears possible by means such as EM/radioactive radiation, is problematic. Indirectly first destroying the shell, then disrupting the enveloped genomic material while allowing antigen survival, such as spike protein antigens, is the direction to which this invention is directed.

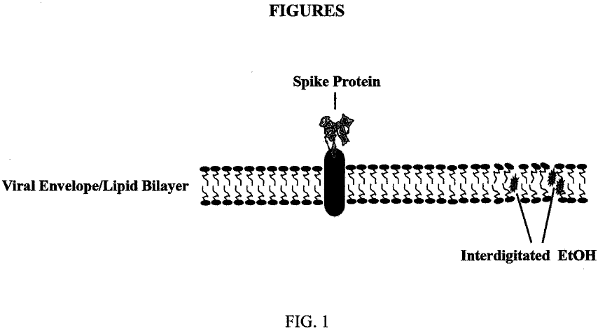

The coronavirus that causes COVID-19 and SARS are virulent, contagious, small/“invisible.” However the outside shell from which the spike proteins extend is considered to be very weak being made of lipid. See FIG. 1. Resources include germicidal agents that are very effective against the weak coronavirus shell, like detergent, bleach and (30-70%) alcohol, but these are also toxic to humans. See for example “Hand Sanitizers: A Review on Formulation Aspects, Adverse Effects, and Regulations,” Jane Lee Jia Jing, Environ Res Public Health. 2020 May; 17(9): 3326, hereby incorporated by reference.

As best understood per the CDC the most feasible explanation for the antimicrobial action of alcohol is denaturation of proteins, but as now best understood there is no information of ethanol activity against glycoprotein spikes in coronavirus. A coronavirus has a diameter of 120 nm, which is 267 times larger in diameter than an ethanol molecule (0.450 nm). Therefore, an object of this invention is to take advantage of the Brownian motion of the small ethanol molecules with water to rupture the large coronavirus lipid shell that then inactivates the genome or genetic material while allowing as many glycoprotein spikes to remain and exposing any antigenic material within the envelope/shell. The effectiveness of such a vaccine is premised on the characteristic that the residual coronavirus spikes and the recognizable antigenic material will interact with the host cells to promote generation of antibodies in the absence of infective agents.

Certain embodiments of this invention provide a means for inactivating the genome encapsulated in the spherical body of the coronavirus associated with COVID-19 by penetrating the shell instead of the spikes which extend therefrom. It is understood that the genome or RNA located within the shell is the infectious agent after the spikes or keys have engaged the receptors on the cell to attach a virus to a host cell, and is antigenic but “hidden” from the immune response by the shell, unless this shell is ruptured or broken apart.. However, it is also currently understood that the spikes themselves which are recognized by the host human body, and which cause the human body to generate antibodies to fight the disease. Thus if the genome or RNA can be destroyed without destroying all of the spikes, antibodies will still be generated without the introduction of the harmful RNA or genome or genetic material which is the infectious agent, then a vaccine can be created without the danger of infection when introduced into the human body.

One material that can be used to penetrate, disrupt and/or rupture the shell and deactivate the genome is ethanol/ethyl alcohol.. Other alcohols such as methanol or propanol are also germicidal against corona viruses but ethanol is believed to be to most effective. Ethanol is both germicidal and nontoxic for consumption. Recent studies show ethanol effectiveness at a 30% concentration by weight (or_60 proof alcoholic beverage) for deactivating the genome and can comprise an effective dose. The only potential drawback in using ethanol is a potentially deleterious effect on the spike proteins; however, current literature online and Pubmed (medical studies) show the glycoprotein spikes are not or only minimally affected by ethanol. Preliminary testing has confirmed treating an egg (glycophosphoprotein) in 50% rubbing (isopropyl) alcohol generates no visible change (i.e., no denaturing or reducing the biological activity), but higher concentrations and longer exposure times are known to denature or “cook” the glycophosphoprotein/egg, thereby supporting the need for the lowest possible ethanol concentration and exposure time to inactivate the coronavirus while allowing the survival of spike protein epitopes. Even if ethanol denatures/incapacitates some spike proteins that are essential in starting the immune process, the ethanol molecule is believed to leave sufficient number of spikes intact from lipid-ethanol attraction away from the spike proteins and random Brownian-type motion.

Ethanol may also be used to impair a pathogen for use in the vaccines of the present invention. In such situations, the ethanol treatment may impair the pathogen’s ability to cause disease (permanently or temporarily). Such impairment may include rendering the pathogen replication incompetent without killing it. The impairment, in certain embodiments, may be reversible, or partially reversible, such that the pathogen may recover some ability to replicate after the elimination or reduction of the ethanol. In certain embodiments, this reduction in impairment may be a function of the recipient organism’s normal bodily functions. For example, removal/reduction by normal metabolism as the treated pathogen of the vaccine passes through the gastrointestinal tract of animal recipient-thereby presenting the pathogen (or increase numbers of the pathogen to the host immune system after bypassing area of the body that are more susceptible to infection in favor of presentation in less vulnerable areas such as the lower GI tract.

Coronaviruses are enveloped, single-stranded RNA viruses, which means that their genome consists of a strand of RNA (rather than DNA) and that each viral particle is wrapped in a protein “envelope.” Viruses all do basically the same thing: invade a cell and co-opt some of its components to make many copies of themselves, which then infect other cells. But RNA replication typically lacks the error-correction mechanisms cells employ when copying DNA, so RNA viruses make mistakes during replication. Coronaviruses have the longest genomes of any RNA virus-consisting of 30,000 letters, or bases-and the more material a pathogen copies, the more opportunity there is for mistakes. The upshot is that these viruses mutate very rapidly. Some of these mutations may confer new properties, such as the ability to infect new cell types or even new species.

The encapsulated coronavirus ball is made of a lipid (fat) envelope and (membrane) protein. It is common knowledge that fat does not mix with water (i.e., coronavirus shell remains intact), so we use soap/detergents to clean pots/pans for cleaning, where soaps essentially make fats “soluble in water because the fats are attracted to the non-polar tail part of the soap while the polar head makes the whole complex (soap and fat molecules) dissolve in water.” Ethanol is an organic solvent that readily dissolves lipids that are non-polar organic compounds (but where lipids are insoluble in water), and would readily dissolve the lipid coronavirus shell, and break it apart.

Nucleic acids (DNA, RNA) are soluble in water; therefore, a water-diluted solution of alcohol/ethanol (i.e., 30-70%) is necessary (vs 90+ percent) as a germicidal agent to then essentially dissolve the nucleic acids from the viral shell, further breaking apart the virus.

Water is a polar molecule - it has a partial negative charge near the oxygen atom due the unshared pairs of electrons, and partial positive charges near the hydrogen atoms. Because of these charges, polar molecules, like DNA or RNA, can interact electrostatically with the water molecules, allowing them to easily dissolve in water. Polar molecules can therefore be described as hydrophilic and non-polar molecules, which will not readily interact with water molecules, are hydrophobic. Nucleic acids are hydrophilic due to the negatively charged phosphate (PO3-) groups along the sugar phosphate backbone.

The glycoprotein spikes and other viral proteins would be substantially unaffected in preparing this expedient vaccine when using the lowest possible ethanol concentration (e.g., 5-30%) in the shortest possible time to inactivate the coronavirus (e.g., 20 -30 sec per recommended germicidal times) for several reasons:
(1) In general, not all alcohols denature proteins, and ethanol is often used to denature proteins, where the effect of ethanol is not consistent for all proteins and where there is a temperature effect. In recovering nucleic acids (without changing structure), “ethanol precipitation is a commonly used to technique for concentrating and de-salting nucleic acids (DNA or RNA) preparations in aqueous solution;” therefore, ethanol in such a de-salting technique should be beneficial to help preserve spike proteins (after viral inactivation).
(2) Rubbing alcoholic will denature or “cook” an egg white (glycophosphoprotein), more noticeable with higher concentrations over a longer time (an hour).
(3) Statistical (math/physics) modeling (using sizes, quantities, etc) supports this concept.

Ethanol treatment of the pathogen for use in the present vaccines may be at any ethanol concentration/time that provides the desired effect on the pathogen (i.e., inactivation or impairment). In certain embodiments, ethanol concentrations and contact times (at room temperature) to inactivate, attenuate, or impair the coronavirus, or other pathogens, (that will then allow the maximum number of spikes/epitopes to be sufficiently unaltered) may be between about 1% to about 90% by weight (more preferably between about 10% to about 80% by weight for between about 10 to 120 seconds. In still other embodiments, ethanol inactivation may be accomplished at concentrations ranging from about 30% to 70% by weight ethanol. In preferred embodiments 30% ethanol by weight in contact with the coronavirus, or other pathogens, for 20-30 sec. See also “Inactivation of Severe Acute Respiratory Syndrome Coronavirus 2 by WHO Recommended Hand Rub Formulations and Alcohols,” Annika Kratzel, Emerg Infect Dis. 2020 Jul; 26(7): 1592-1595, hereby incorporated by reference.

In certain embodiments, the present invention provides vaccines against corona viruses, or other similar viruses, that include ethanol inactivated viral organisms or parts thereof. In still other embodiments, the vaccines of the present invention may also contain ethanol treated cell cultures, viral contaminated donor samples, or combinations thereof. One of skill in the art will readily understand that moieties other than ethanol may be used to inactivate the viruses, or virus containing samples, and that the use of such other moieties is within the scope of the present invention. One will also understand that the corona virus, as used herein, is an exemplary embodiment and that the invention encompasses vaccines containing other pathogens inactivated according to the procedures provided in this disclosure.

In certain embodiments, ethanol (or a similar inactivating agent) may be used to attenuate or impair the target pathogen for incorporation into the vaccine by making the pathogen replication incompetent. Live-Ethanol impaired oral vaccines may be effective as live-attenuated ones for oral administration to elicit both broad and robust immune responses.

Certain embodiments of the vaccines of the present invention may also include packaging of the inactivated virus or virus along with known stabilizers, adjuvants, antibiotics, preservatives, coatings, gels, and combinations thereof.

Manufacturing can be performed in any appropriate setting including regulatory-agency-approved facilities or even in a hospital/clinic (with controlled access, using PPE) by simply adding the ethanol of predetermined concentration to a determined amount of virus in an otherwise sterile test tube, capping and swirling the tube, then diluting it by pouring into sterile water or drink (to effectively minimize any further potential spike denaturing/degradation).

Administration of the vaccine can be accomplished by any suitable route of delivery. In certain embodiments, the vaccine may even be administered within the predetermined time for inactivation by allowing the patient to immediately drink the liquid or by immediate dilution for drinking at a later time. Significant dilution maysignificantly reduce any potential spike inactivation or degradation and subsequent vaccine effectiveness. Storage of the vaccine under low temperatures may also prevent or delay any potential ethanol degradation of the spikes until needed for administration. With oral administration of ethanol inactivated pathogens such as viruses, gastric acids may act synergistically with the ethanol to further increase anti-virucidal activity. In still other embodiments, the vaccine of the present invention may be administered via rectal suppository.

In certain embodiments, the vaccine(s) of the present invention may be administered by a combination of routes simultaneously or sequentially. For example, the use of both oral and injected administration of an ethanol inactivated vaccine would be complementary–oral administration first would mount an immune response and therefore, reduce the risk from the second administration by injection while bolstering the immune response.

For pediatric cases or individuals who do not consume alcoholic beverages for religious purposes, separation of the ethanol and the inactivated virus can be done very readily, such as by slight heating and evaporation under controlled/vented-type environment for safety purposes, or (ethanol) precipitation and centrifugation. Too much drying will denature proteins. Water or some other fluid may then be added to the residual inactivated virus for oral consumption. The “dried” virus may also then be processed in sterile fashion by adding normal saline and/or stabilizing adjuvant for IV injection.

Inactivation (killing) of the virus prevents infection/side effects, and oral (PO) administration would not reasonably create any lung infections, taking advantages of both Salk and Sabin vaccines simultaneously. The ACE-2 receptors targeted by coronavirus, are predominantly in the lungs, but also exist in the intestines, where the very low acidity in the stomach is hostile to virus survival.

Vaccines that are orally administered may be broken down by stomach acid down the medication ingredients. However, gel pills will may safely escort the medication through the stomach and deposit the medication in the intestines. Recent research has shown that an influenza vaccine is gel pill form does suffer from acid degradation. Therefore, the use of pills with a protective (i.e. gel-type) shell will be useful to increase spike survival through the highly acidic stomach since research has shown that an influenza vaccine in gel pill form does not suffer from acid degradation.

Although the currently preferred embodiment of this invention is directed to the use of ethanol/ethyl alcohol, this invention is not so limited and other embodiments are within the scope of this invention. Examples are other alcohols (e.g., propanol, butanol, pentanol and hexanol), Hydrogen Peroxide (H2O2), Beta-propiolactone (BPL), salts, sugar, chlorhexidine, copper (cupric oxide and cuprous oxide), povidone-iodine, chlorinated water, quaternary ammonium compounds, the use of extreme pH’s and/or temperatures, high intensity lights (i.e. UV, LEDs, HID), electric charges and or electric current, and electromagnetic radiation of exposure to radioactive material.

What is claimed:

1. A method of generating a vaccine against a pathogen comprising:
a. obtaining a sample of the pathogen;
b. treating the pathogen sample with ethanol to inactive the pathogen.

2. The method of claim 1, further comprising removing the ethanol.

3. The method of claim 1, wherein the pathogen is a corona virus.

4. The method of claim 2, wherein the corona virus is SARS-CoV-2.

5. The method of claim 4, wherein the pathogen is treated with ethanol at a concentration of between about 30% to 70% weight for a period of about 30 seconds.

6. The method of claim 4, wherein the SARS-CoV-2 virus is obtained from a patient sample.

7. The method of claim 4, wherein the SARS-CoV-2 virus is obtained from a tissue culture.

8. A vaccine comprising:
a. an ethanol inactivated pathogen; and
b. a pharmaceutically acceptable carrier.

9. The vaccine of claim 8, wherein the pathogen was inactivated by treatment of the whole pathogenic organism by treatment with ethanol at a concentration of between about 30% to 70% weight for a period of about 30 seconds.

10. The vaccine of claim 8, wherein the pathogen is a corona virus.

11. The vaccine of claim 10, wherein the corona virus is SARS-CoV-2.

12. The vaccine of claim 11, wherein the SARS-CoV-2 virus is obtained from a patient sample.

13. The method of claim 11, wherein the SARS-CoV-2 virus is obtained from a tissue culture.

14. The vaccine of claim 11, further comprising a stabilizer, an adjuvant, an antibiotic, a preservative, or any combinations thereof.

15. The vaccine of claim 8, wherein the vaccine is inactivated by rendering it replication incompetent.

16. The vaccine of claim 8, wherein the vaccine is inactivated by attenuation.

17. A method of vaccination an organism against a pathogen comprising:
a. obtaining a vaccine comprising a whole pathogen that has been ethanol inactivated to render it replication incompetent and functionally disrupt any lipid bilayers; and
b. inoculating the organism with the vaccine.

18. The method of claim 17, wherein the pathogen is a corona virus.

19. The method of claim 18, wherein the corona virus is SARS-CoV-2.

20. The method of claim 19, wherein the organism is inoculated via oral administration of the vaccine.

21-27. (canceled)
